(19) United States
(12) Patent Application Publication
(10) Pub. No.: US 2015/0164675 A1
(43) Pub. Date: Jun. 18, 2015

(54) LIMB FIXATION OR RESTRAINING DEVICE

(71) Applicant: Yoel Schlesinger, Brooklyn, NY (US)

(72) Inventor: Yoel Schlesinger, Brooklyn, NY (US)

(21) Appl. No.: 14/568,344

(22) Filed: Dec. 12, 2014

Related U.S. Application Data
(60) Provisional application No. 61/915,261, filed on Dec. 12, 2013.

(51) Int. Cl. A61F 5/058

(52) U.S. Cl. CPC A61F 5/05858

(57) ABSTRACT

A device and method for the fixation, support traction, or restraint of limbs or body parts, into immobile positions with substantial prevention of movement from the immobile position. The device includes at least two fixation elements configured to be attached to different sites on a patient's body to hold the device on the patient's body wherein the fixation elements are maintained in a substantially fixed distance relation with respect to each other with an adjustable length connection element. The connection element is configured to be one of length adjustable or adjustably connected to the fixation elements to provide a desired distance between the fixation elements. The device is further configured to substantially lock the desired fixed distance against unwanted dislocation or change of position.

### FIELD OF THE INVENTION

This invention relates to splints and fixation or restraining devices for preventing movement of limbs or other body parts of living beings including animals and humans and particularly to devices for position fixing of arms and legs around elbow and knee joints.

### BACKGROUND OF THE INVENTION

The current method and device for fixation of injured limbs such as after an accident to prevent aggravation or additional injury and pain is by means of a splint and two tying cloths known as cravats. For example, the splint is placed across a forearm and upper arm around an elbow for an arm injury. Similarly, the splint is placed across a thigh and shin around a knee for a leg injury. Once the splint is positioned and while arm or leg is maintained in position, ends of the splint are tied with the cravats by utilizing a defined winding and tying procedure to provide both a secure hold and avoiding undue pain to the injured person. Such fixation requires training for proper deployment and involves inordinate amounts of time while an injured person must painfully maintain a non-moving position. The training is standard for EMT and hospital personnel and often requires viewing of videos or hands-on lessons.

### SUMMARY OF THE INVENTION

It is accordingly an object of the present invention to provide a device and method of use for injured limb or body part fixation for animal or human patients, which requires little or no training for proper and rapid secure deployment.

Generally the present invention comprises a compact and easily carried and deployable device and method for the fixation, including support traction, of body parts, such as arms and legs into immobile positions with prevention of movement around a joint, in the event of an injury or other untoward event. The prevention of movement is often necessary to prevent aggravation of the injury and possibly to prevent further bleeding and tissue damage.

The device comprises at least two fixating or fixation elements configured to be attached to different sites on a patient's body to hold the device on the patient's body. The fixation elements are maintained in a substantially fixed distance relation with respect to each other with an adjustable length connecting or connection element, which is configured to be length adjustable or adjustably connected to the fixation elements to provide a desired distance between the fixation elements. The device is further configured to substantially lock the desired fixed distance against unwanted or unintentional dislocation or position change (but which allows for intentional change). Locking, as used herein, is a fixing of position, effected either with a manual operation or with the use of materials which sufficiently resist movement, to maintain the limb or body parts in a desired separated relation against movement or dislodgment pressure which may be exerted by the patient. Alternatively, if used for restraining of body parts relative to each other, the length is adjustable to maintain the body parts adjacent each other. Splinting or traction is used to simply limit aggravating movement of an injured body part.

As non-limiting examples, in one embodiment the fixation elements are size-adjustable clamps, configured to be conformed to limbs or body structure. In another embodiment, the fixation elements are wrap-around bands, which more flexibly conform to the limbs or body structure. The connection element is connected to each of the fixation elements with either a fixed connection with the connection element being configured to be changed in length between the fixation elements or with the fixation elements being movable relative to the connection element for the adjustable length connection. The connection element, whether being connected with a fixed connection or movable connection may be provided with a disengageable connection with the fixation elements such as for storage break-down. The connection between the fixation elements and the connection elements is specifically not with simple ties which include the limbs or body parts.

The device is utilized in a splinting, fixation, traction or restraining of body parts or limbs with the steps of:
connecting at least two fixation elements to a connection element,
adjusting the length of the connection element between the fixation element to a desired length sufficient to maintain body parts or limbs apart from each other or restrained adjacent each other against movement away from the desired length,
engaging the fixation elements with body parts or limbs to be splinted, fixed or restrained against movement, and locking the fixation elements and connection elements against undesired relative movement. Traction involving a single body part is included herein as preventing relative movement of the body part itself.

The sequence of the above steps may be varied such as with the entire device being configured and locked prior to placement on the body parts or limbs or with the sequential placement of the fixation elements and connection elements and locking therebetween.

Also included within the definition of locking, as used herein, is an inherent rigidity of the connection elements, which resists untoward patient movement or dislodgement of the device.

The above and other objects and features of the present invention will become more evident from the following discussion and drawings in which:

### SHORT DESCRIPTION OF THE DRAWINGS

FIG. 1 depicts a first embodiment of the limb fixation device of the present invention as applied to an arm around an elbow;

FIG. 2 depicts a second embodiment of the fixation device wherein a locking joint provides the positioning variation needed for placement;

FIG. 3 depicts the limb fixation device of FIG. 1 as applied to a leg around a knee, with a second fixation device shown in dashed lines on the opposite side of the leg as an optional bracing element;

FIG. 4 depicts another configuration of the splinting element with two slots at the ends thereof;

FIGS. 5 and 6 depict two variations of threaded stud elements integrated with the fixation bands;

FIG. 6a shows a variation of the bolt of FIGS. 5 and 6;

FIG. 7 shows the splint element as placed on the studs of two open band elements with hook and eye fastening ends;

FIG. 8 is a view of a slotted splint element with shaped teeth forming multiple position shaped apertures for engagements with studs to preclude necessity for sliding adjustment;

FIG. 9 is a variation of the slotted splint element of FIG. 7, with dual slots;

FIG. 10 depicts a variation of the slotted splint element of FIG. 7 with a hinged connection between segments of the splint element;

FIG. 11 depicts a pair of limb fixation devices interconnected with a rotational connector element;

FIG. 12 illustrates another embodiment of the splint element constructed of interfitting pipe like elements including angular and straight elements;

FIG. 13 alternatively illustrates slotted elements configured for interconnection into a desired length;

FIG. 14 depicts another embodiment of the splint element structure with an accordion-like four bar variable length configuration;

FIG. 15 is another embodiment of the splint element connector structure with multiple apertures for distance selection;

FIG. 16 is a further embodiment of the splint element connector structure movable rotational wheel structures within an elongated channel element;

FIG. 16a depicts a limb fixation device with the splint element connector of FIG. 16;

FIG. 17 depicts a splint element connector embodiment with elongated adjustable-length, nested, sliding elements;

FIGS. 18, 18a and 18b show embodiments with articulation joints as part of the splint element connector;

FIG. 19 shows a movable compass like configuration of the splint element connector;

FIGS. 20 and 21 respectively show three and four piece rod configurations of another embodiment of the splint element connector;

FIGS. 22a and 22b show alternative configurations for the limb fixation device as a configurable and lockable clamp element;

FIG. 23 show a fixation device with clamps and articulated jointed elements of the splint element connector;

FIG. 24 depicts a variable length pipe-like configuration of the splint element connector connected to clamps with tightenable ball joints;

FIG. 25 shows another embodiment of the limb fixation device with tightenable clamps connected by a flexible pipe or tube with resistance to displacement movement;

FIGS. 26a and 26b depict a limb fixation device with clamps and a spring loaded connection element and as used to maintain parts of an arm separated around an elbow;

FIG. 27 depicts a limb fixation device of another embodiment with multiple interconnection adjustment elements of the connector element;

FIG. 28 depicts a limb fixation device as provided with neck and body sling supports;

FIG. 29 shows another embodiment of the limb fixation device with clamp fixation element and a connector adjustable with arc movements on circular track segments;

FIGS. 30a and 30b depict embodiments of the limb fixation device as utilized as straight double sided and single sided traction splints respectively;

FIG. 31 is another embodiment of the fixation device as used in the limb fixation devices with a strap or buckle restrainer clamp; and

FIG. 32 is a slidingly adjustable connector element similar to that of the embodiment in FIG. 17 with overlapping slotted elements for insertion of locking elements such as nuts and bolts.

### DETAILED DESCRIPTION

In a first embodiment, the device comprises two wrap-around bands, fixation elements, with each being of sufficient dimensions to encircle a limb therewith and to be fastened thereon. Variable fastening elements such as adjustable hook and eye fasteners or a belt buckle structure are integrated with the bands to ensure a tight fastening in place on the limb. The bands in this embodiment are each configured to be positioned on an arm or leg limb on opposite sides of the joint (elbow or knee) with each band being integrally provided with an outwardly extending stud element or other type of position fixable extension element such as a clamp. The bands are further configured to be positioned on the limb such that the extension such as stud elements extend in the same upper or lower direction relative to the limb and are generally parallel with each other. The device further comprises a connection element in the form of an elongated rigid longitudinally extending rod or splint element such as of a flat, ruler-like configuration to facilitate limb placement and locking, as will be described.

The splint element, in this embodiment comprises an integrated structure configured to fixedly hold the stud or other extension from the band to provide an adjustable fixed connection between the band and the splint element. In another embodiment the structure is configured with a longitudinally extending slot in the splint element to effect the holding of the stud. The longitudinal slot, is of sufficient length to span the distance between bent parts of the limb around the joint (either elbow or knee) or even to maintain the limb in a straight position as a common splint. With a straight position configuration, the device need not span a joint and may simply be used as a substitute for a splint on either side of a joint. The splint element comprises at least one elongated slot or separated slots at the ends thereof sufficiently sized for the insertion of the studs of the bands therethrough.

In another embodiment the splint element is jointed with one or more joint locking elements and may be reduced in size for carrying efficiency in a configuration similar to that of a folding wooden ruler with locking joints. In such embodiment the joint or joints provide the adjustment variation wherein the slots may be replaced by simple apertures. As a further variation, the splint element or connector may be comprised of numerous jointed elements in an articulable configuration with each of the joint connections being lockable in order to achieve a desired fixed distance between limb or body fixation elements.

In a still further embodiment, the slot may be replaced with a retaining track or other structure, which provides a cooperative fixed engagement between the splint and the bands. Alternatively, any other element may be utilized which is integrated with the band and is able to be fixedly clamped to the splint.

The stud elements of the bands are of sufficient length to extend at least partially and preferably substantially fully through the slots or apertures for engagement with a locking element such as a cap or locking nut. To enhance holding between the stud and the splint element it is preferred that the base of the stud which engages the splint element, be rounded.

In operation, the limb is arranged in the fixation position, the bands are placed around the forearm-arm or thigh-shin across the elbow or knee, as the case may be, with the respective studs positioned to extend in the same direction and generally parallel with each other. The slotted splint element is placed diagonally across the joint and positioned such that the generally parallel studs are each positioned within a corresponding adjacent slot or aperture in the splint element. If necessary, particularly with respect to leg injuries, it may be desirable to provide splint elements on both sides of the injured leg with the opposite side splint elements being engaged with separate pairs of band.

In yet another embodiment, the studs are threaded and the locking element comprises a locking nut, which compressingly engages with the splint surface. Alternatively, the slots may be provided with shaped retaining elements to hold the studs against transverse movement with a cap placed on the stud to prevent vertical disengagement.

For patient comfort it is further desirable that the studs be retained within the thickness of the bands or with the base being flush with the inner surface of the bands, which engage the skin of the injured person.

In other embodiments, the connecting element may be comprised of numerous sized snap together elements or interfitting pipe joints, which are selectively connected to provide the requisite fixed distance between fixation elements. Telescoping elements with position locking elements such as spring load pins and apertures, such as used with canes and crutches provides another configuration for a different embodiment of the connection element. Three or four bar interlocking rods form different combinations of configurable connecting elements though often of larger bulk.

It is understood that the connecting elements, while providing for variations in distance between fixation elements, once they are fixed into a selected length they can be utilized as single or even, such as with secondary interconnection elements, double traction devices or elongated splints. The connecting elements may also be configured with multiple connection points with the fixation elements as well as being hinged or with combination of slots and hinged or rotatable pin connections.

Variations of the fixation elements are similarly possible with tightenable circular elements or straps providing alternative arrangements.

The splinting devices, particularly those utilized with the arms may be further provided with sling elements for assistive support by being draped on the neck of the patient or with body support such as with waist encirclement elements.

For better determination of the fixed distance, the connector element may be appropriately marked with distance markings.

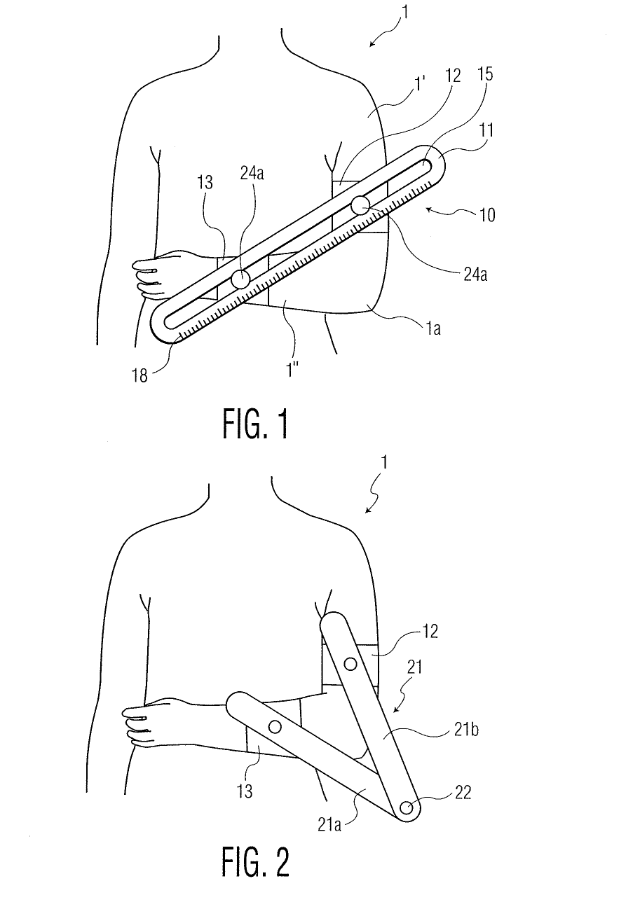

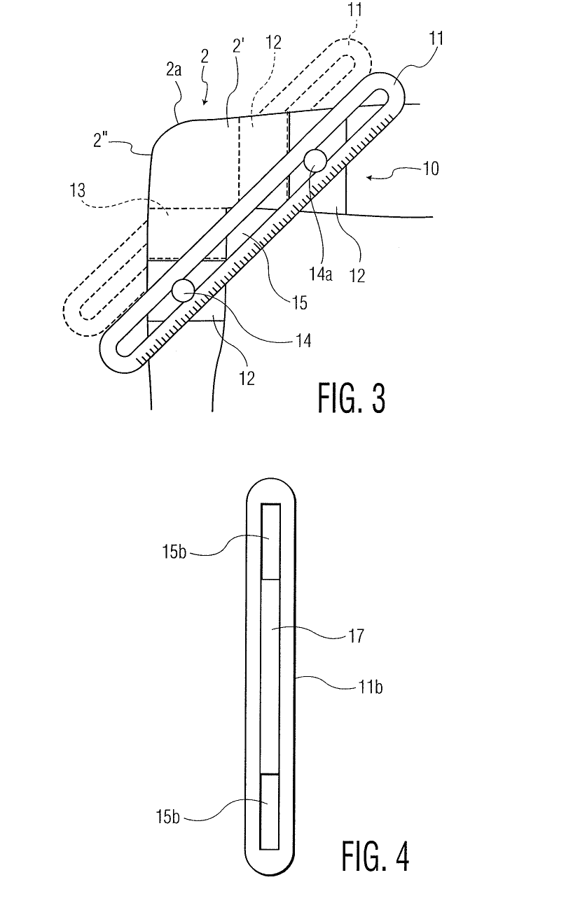

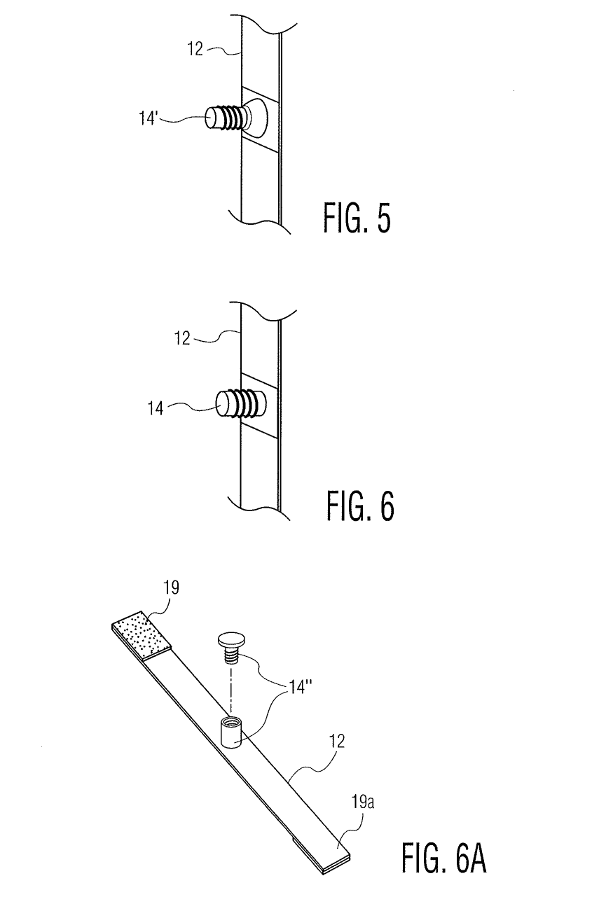

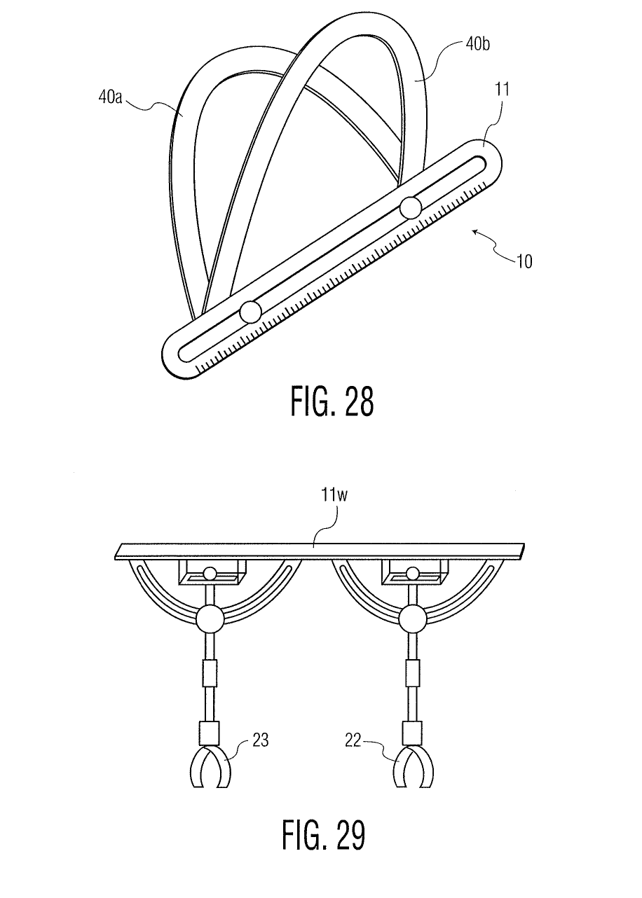

With specific reference to the drawings, FIG. 1 depicts a basic splinting of the arm 1 of a patient between the upper arm 1′ and the forearm 1″ in a right angle configuration around elbow la with limb fixation or splinting device 10. The device 10 is comprised of fixation bands 12 and 13, which are shown wrapped around upper arm 1′ of arm 1 and forearm 1″. The device 10 further comprises connector element 11 with longitudinal slot 15. Stud elements such as 14 and 14′ or 14″ of FIGS. 5, 6 and 6a are integrated with the fixation bands and are each positioned to fit within slot 15 at specific positions within the slot to achieve the desired relative positioning of upper arm 1′ and forearm 1″. Cap nuts 24a and 24 are used to tighten or lock the studs into the appropriate position against movement. Length markings 18 on connector element 11 may be used to assist in determining proper placement of the studs with respect to the connector element. Neck and body sling elements 40a and 40b, as shown in FIG. 28, can be provided to allow for neck and/or body support of the device. FIG. 3 depicts the splinting of a leg 2 with two splinting devices 10 with respective bands 12 and 13 on thigh 2′ and shin 2″ around knee joint 2a. Utilization is similar to that with the arm of FIG. 1 but with a leg being more amenable to placement of a splinting device 10 on opposite sides of leg 2 to provide additional restriction against movement.

Operationally, identical bands 12 and 13 are provided with jointing elements of studs 14, 14′ or 14″ as shown in FIGS. 5, 6 and 6a. As depicted in FIGS. 5 and 6, the studs are threaded bolts with the heads imbedded in the band 12 in FIGS. 6 and 6a. The stud 14″ in FIG. 6a has a reverse interior threading. The studs 14 and 14a, as shown in FIGS. 1 and 3, are adjustably fitted within track 15 of connection element 11 and held in place with a nut (such as 24 or 24a) with internal or external threading as appropriate relative to the stud being used.

FIG. 2 depicts another embodiment of the device as shown in FIG. 1 with a jointed connection element 21 between bands 12 and 13 with movable arms 21a and 21b pivoting around position lockable pivot 22.

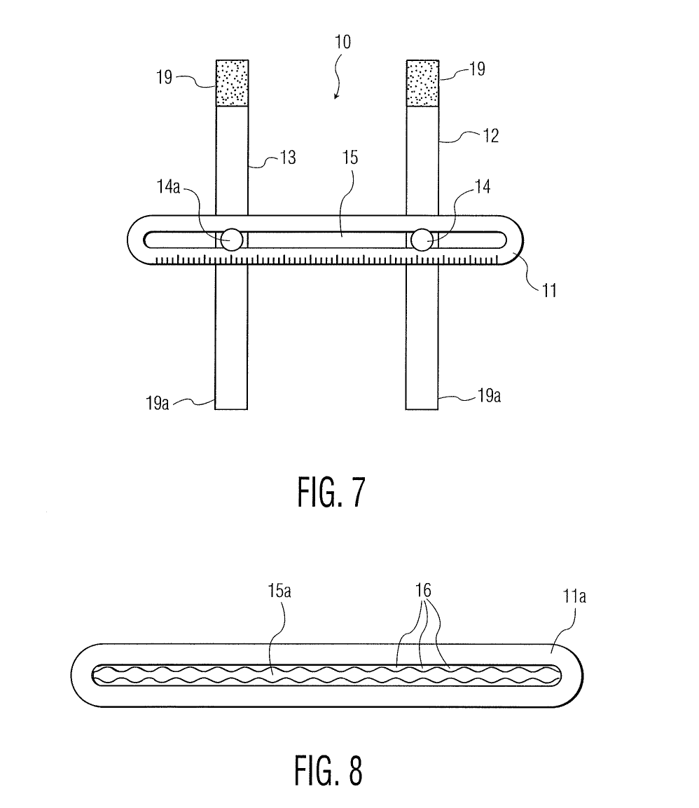

FIG. 7 depicts the device 10 prior to placement of the wrap around bands on the respective limb portions of FIGS. 1 and 3. Hook and eye fasteners 19 and 19a on the bands 12 and 13 enable the rapid deployment and fastening of the fixating bands into non-movable positions on the limbs as shown.

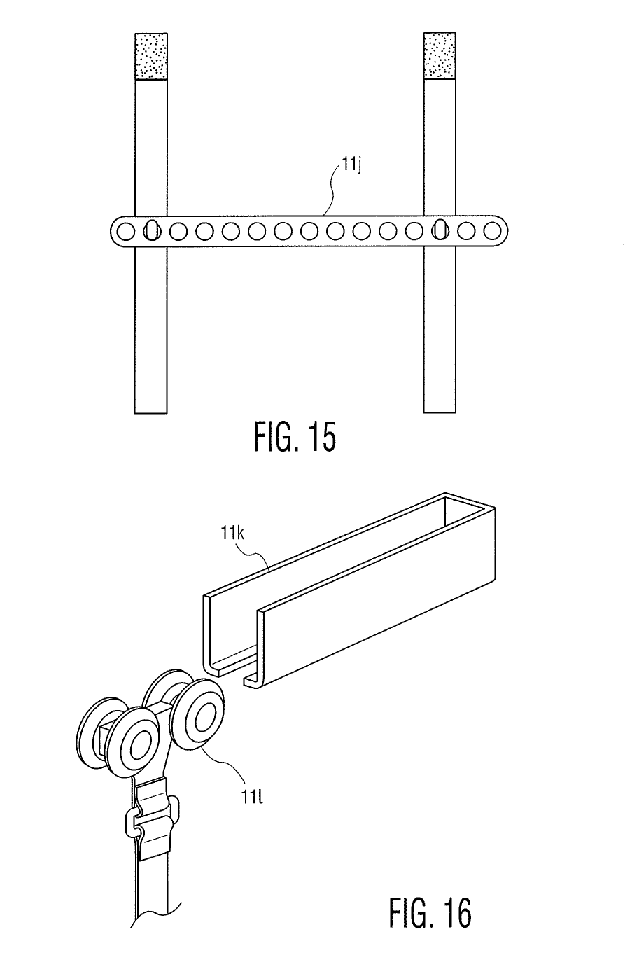

FIGS. 4 and 8 show variations of the connecting elements 11b and 11a with an interrupted slot 15b with solid segment 17 in FIG. 4 and connecting element 11a of FIG. 8 with slot 15a having teeth 16 for more closely holding an inserted stud therebetween and reduction of slippage. FIG. 15 shows a further modification with connecting element 11j being provided with a series of apertures into which the stud can be closely fitted and held in position.

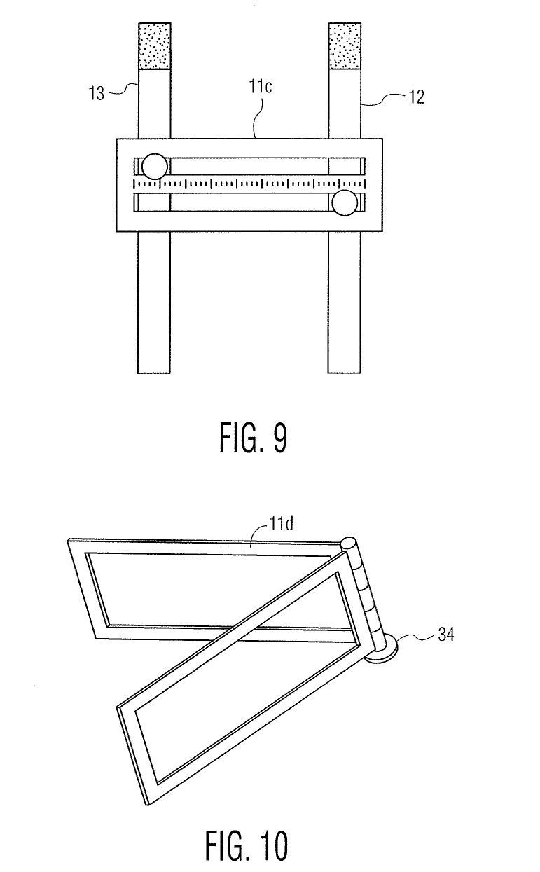

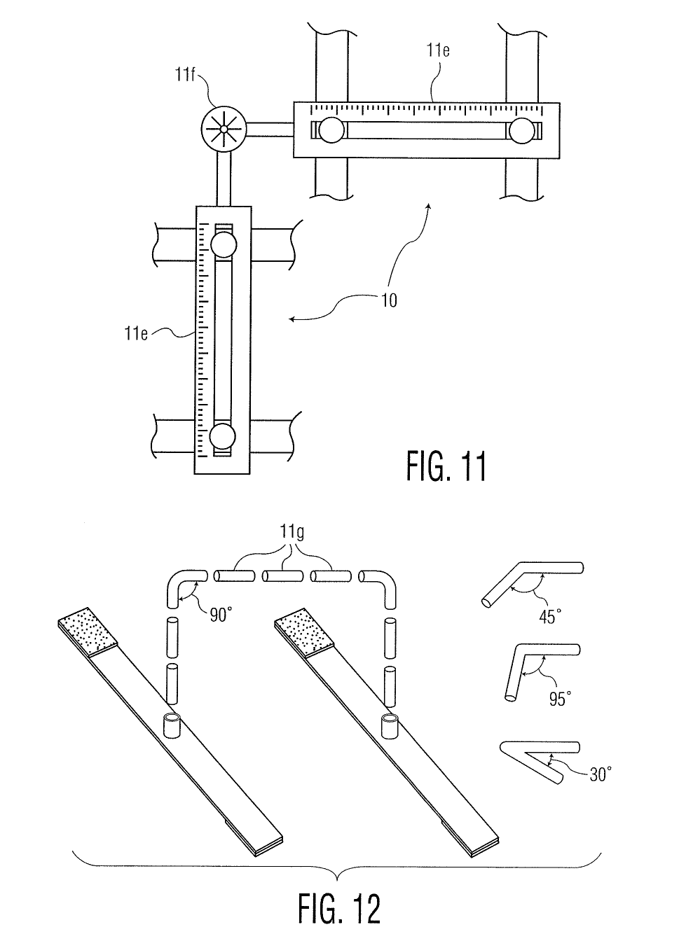

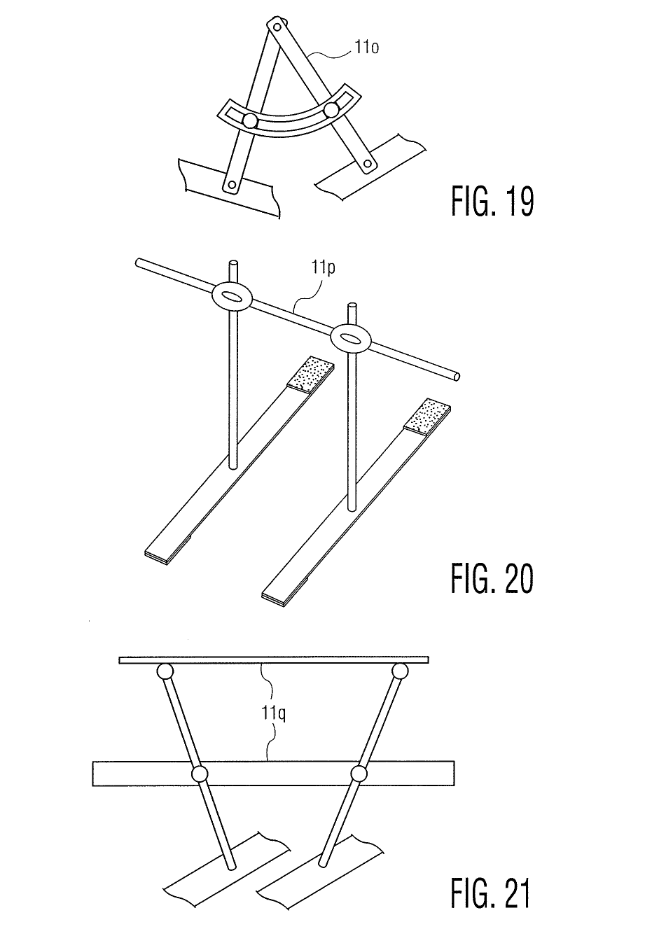

FIGS. 9-21 exemplify various modifications of the connecting element with FIG. 9 showing connecting element 11c with a dual slot structure. Connecting element 11d in FIG. 10 is provided with a hinge structure with a position locking nut 34. FIG. 1 shows a reinforced dual structure of the device 10 with the connecting elements 11e being further movable connected via rotation element 11f.

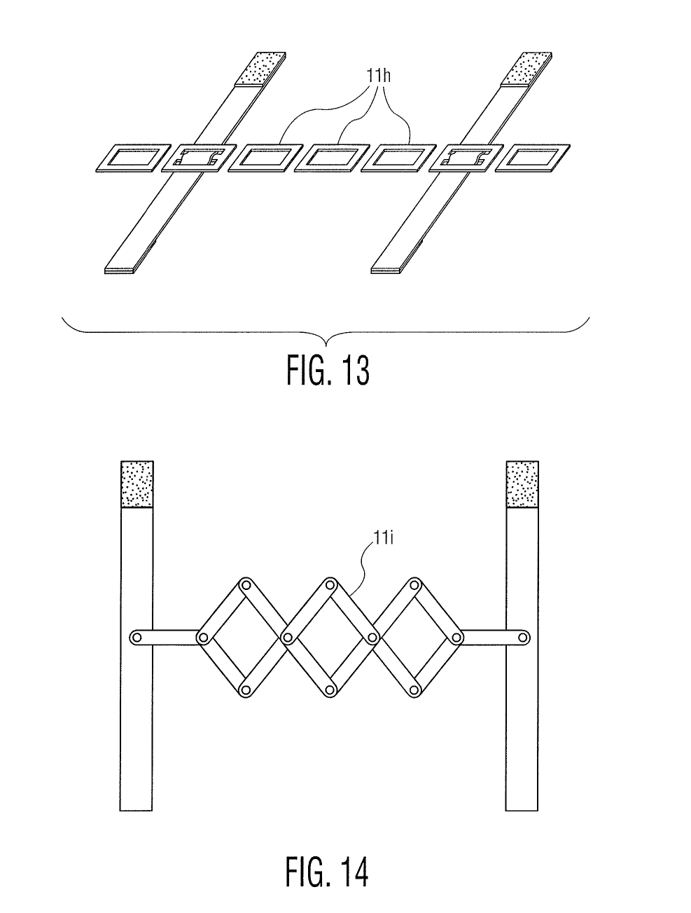

The connecting element embodiments in FIGS. 12 and 13 of 11h and 11g embody interlocking build-up components of snap together elements 11h with an appropriate linear positioning distance as shown being determined by the number of snap together elements. Similarly interfitting pipe elements 11g provide another connection embodiment with bent elbow elements of 30, 45 and 90° permitting configuration variations. In such embodiments, the snap in or connected elements inherently provide the position locking with the placement thereof.

In embodiments wherein the actual length of the connecting element is varied, connection thereof of the fixating elements need not be a movable one and the stud interconnection may be replaced with direct rivet or other connection.

FIG. 14 shows an accordion type extension or compression of connecting element 11i, with locking being effected by appropriate tightening of accordion pivot points.

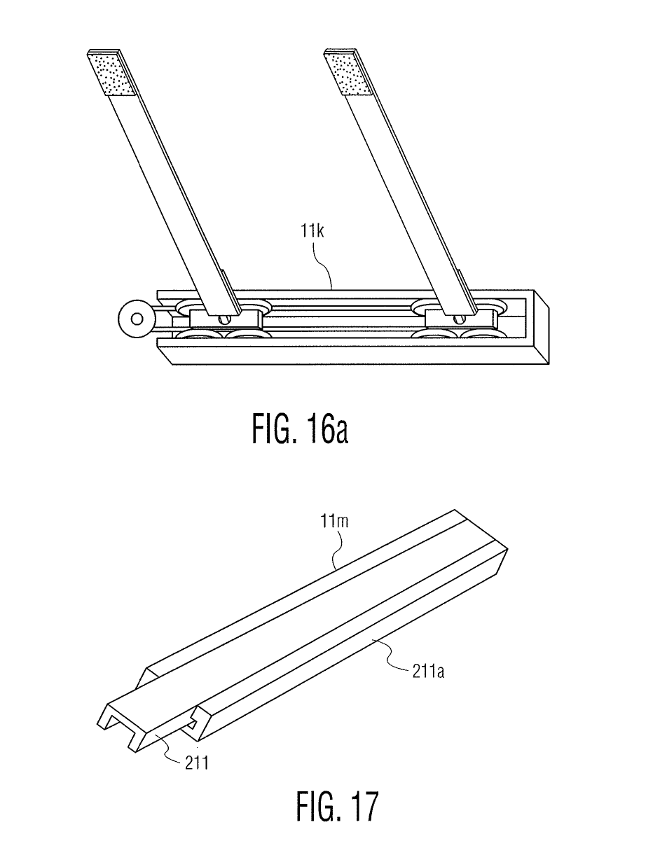

FIGS. 16 and 16a depict a rolling track configuration wherein rollers 11l connected to the fixation elements are movable within a channel track of connecting element 11k to provide the requisite spacing between the fixating band elements. FIG. 17 shows the adjustable connecting element 11m being comprised of sliding sections 211 and 211a.

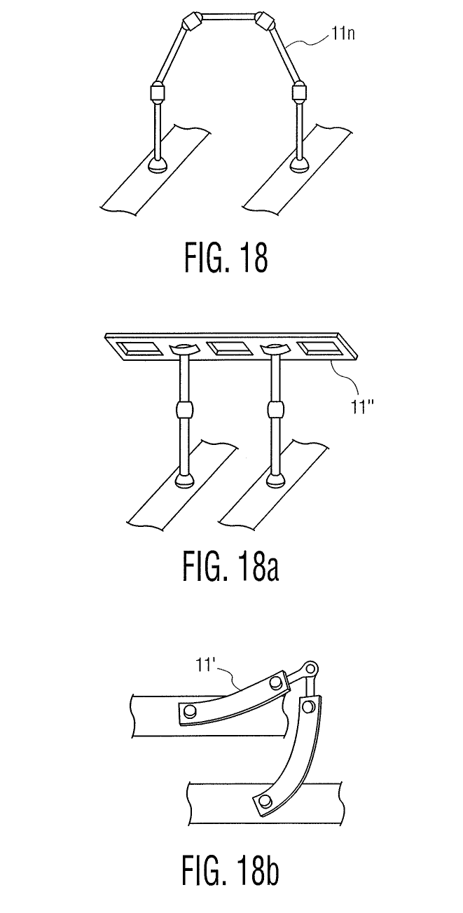

FIGS. 18, 18a, 18b, 19, 20 and 21 depict connecting elements of various embodiments of 11n, 11′, 11″, 11o, 11p, 11q with variations in articulated joints and bars, with each having locking members to lock an articulated distance into place for effective fixation or restraint.

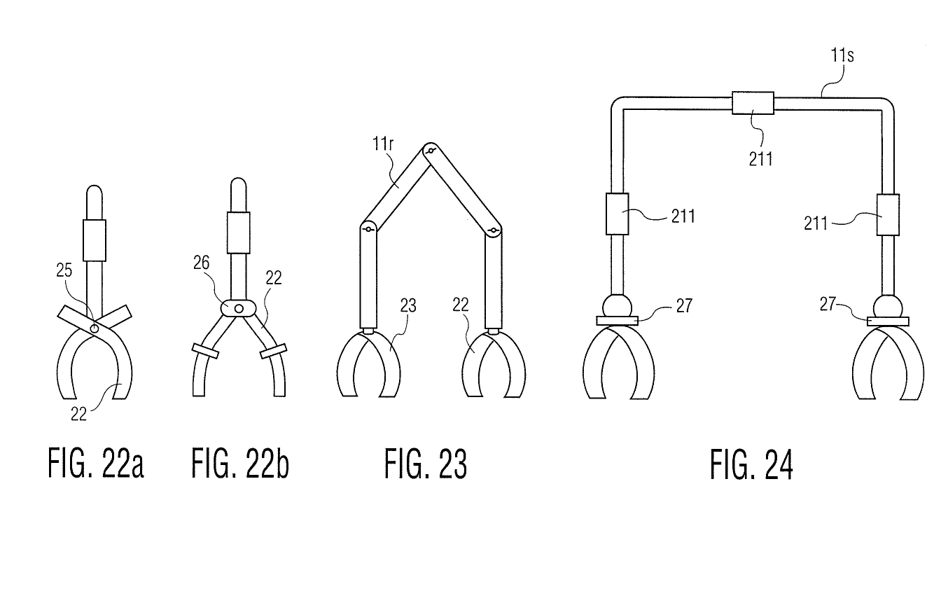

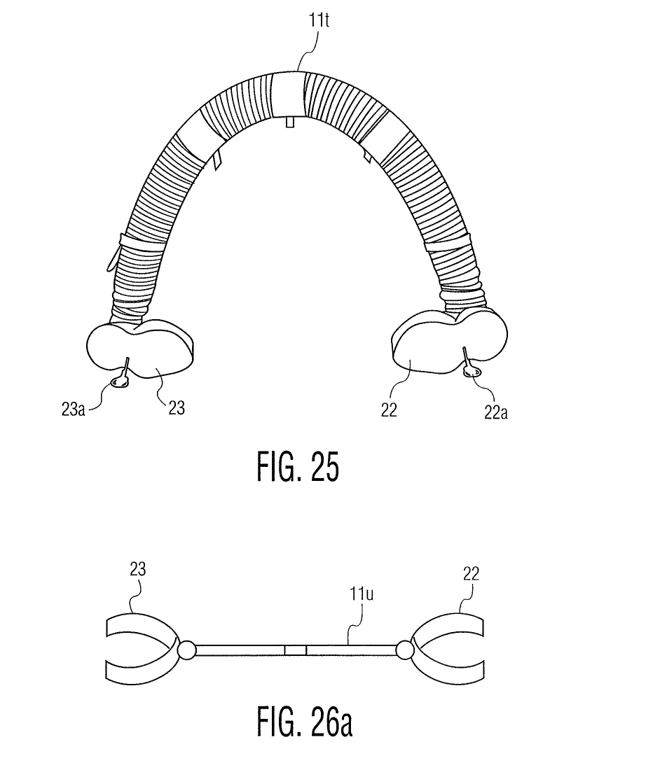

FIGS. 22a and 22b depict clamps 22 as the fixation element with the clamps being adjustable in opening to conform to the shape of a limb or a body part and wherein the clamps are shown with a tightening nut 25 and tightening key 26 to hold the enclosed body part. FIGS. 23 and 24 show different embodiments of the fixation elements 22 and 23 with connecting elements 11r (articulating), 11s with adjustable collar elements 211 and clamp tightening balls 27. FIG. 25 is another embodiment with adjustable clamp elements 222 and 23 with tightening keys 22a and 23a, connected with flexible tube connecting element 11t. The tube 11t requires effort to reshape the arc to provide distance adjustment because of stiffness of the material whereby locking is effected by the very stiffness of 11t.

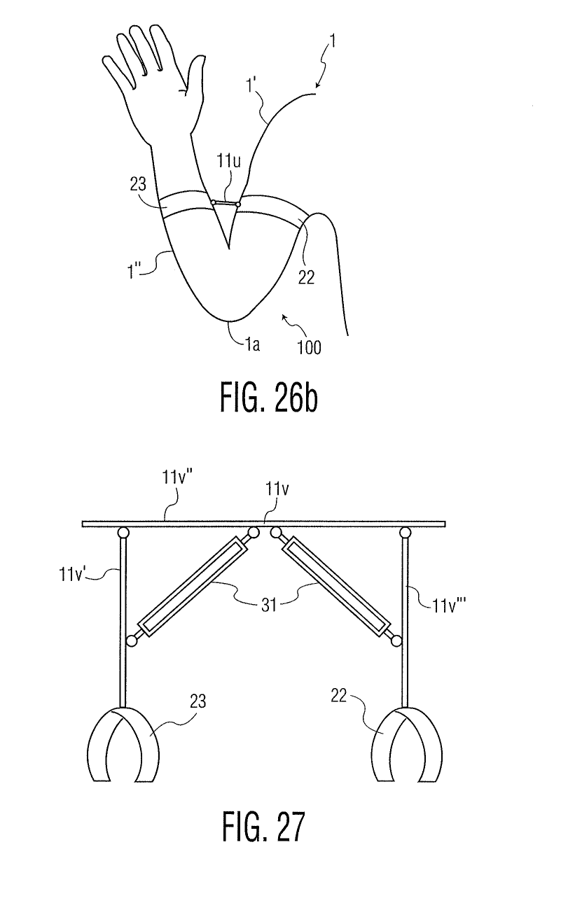

FIGS. 26a and 26b depict an embodiment of the device 100 as used with arm 1 as a restraining element to hold forearm 1″ substantially parallel to upper arm 1′ around elbow 1a with clamps 22 and 23 being held by connecting element 11u.

FIG. 27 shows a reinforced movable and length adjustable structure with clamps 22 and 23 and connecting element 11v being comprised of side bars 11v′ and reinforcing angular elements 31 and cross bar 11v″.

FIG. 29 shows another embodiment of the device with connecting element 11w, clamps 22 and 23 and arc movement for length adjustment.

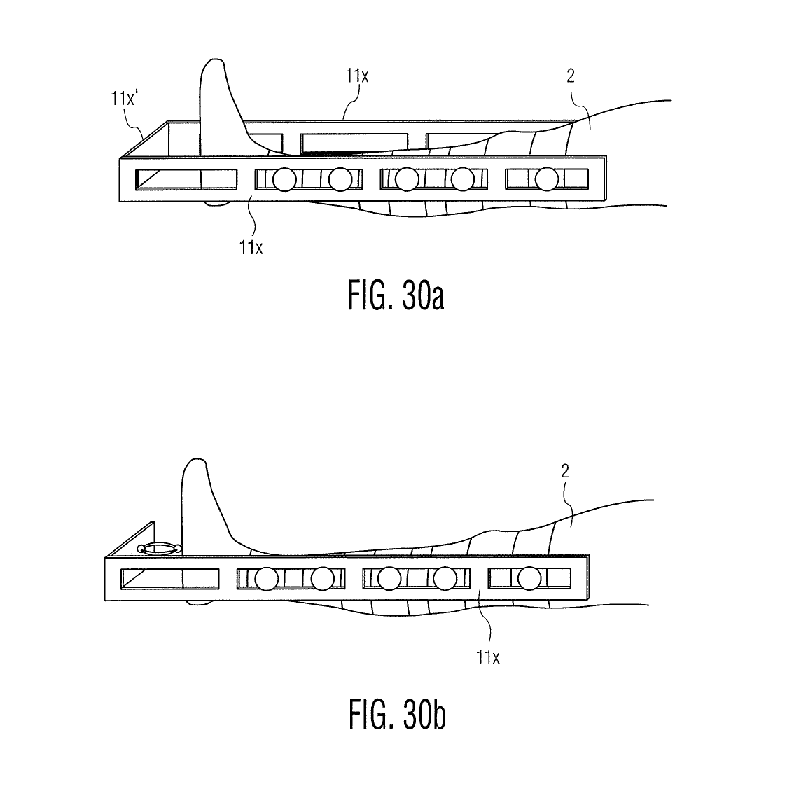

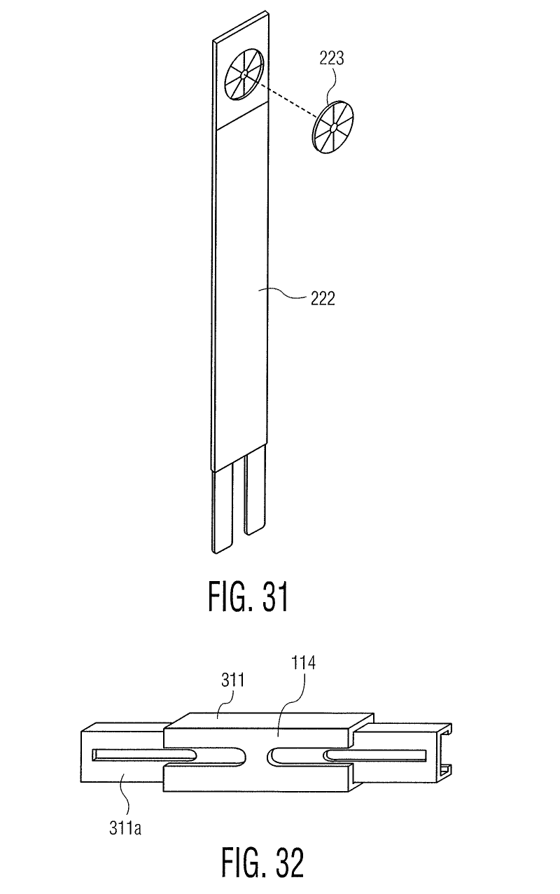

FIGS. 30a and 30b depict connecting elements 11x as being used with fixating element in a straight traction structure with two device being used as shown in FIG. 30a and a single one being shown in FIG. 30b with cross element 11x′ forming part of the connecting structure between the two devices used as shown in FIG. 30a.

FIG. 31 shows a modification of the band structure 222 with straps or buckles being configured as a restraining clamp.

FIG. 32 depicts connecting element 11y, similar to that of FIG. 17 with elements 211 and 211a but with member 311 and 311a with secondary slots for insertion of stud elements and/or locking elements.

It is understood that the above description and embodiments are merely illustrative of the invention and are not construed to be limitations on the present invention. Changes such as in the structure, configuration and interrelation of the fixating and connecting elements, as well as locking elements are possible without departing from the scope of the invention as defined in the following claims.

What is claimed is:

1. A device for the fixation, support traction, or restraint of limbs or body parts, into immobile positions with substantial prevention of movement from the immobile position, comprising at least two fixation elements configured to be attached to different sites on a patient's body to hold the device on the patient's body wherein the fixation elements are maintained in a substantially fixed distance relation with respect to each other with an adjustable length connection element, which is configured to be one of length adjustable or adjustably connected to the fixation elements to provide a desired distance between the fixation elements, the device further being configured to substantially lock the desired fixed distance against unwanted dislocation or change of position.

2. The device of claim 1, wherein the fixation elements each comprise one of a wrap-around band configured to be wrapped around body parts and limbs and fastened thereon and a clamp with an adjustable clamping opening wherein the band and clamp each comprise a member configured to fixedly connect the band or clamp to a connection element having variable length or movably connect the band or clamp to a connection element having a fixed length.

3. The device of claim 2, wherein the band comprises one of a hook and eye fastener and a belt structure configured to effect the holding of the device on the patient's body.

4. The device of claim 2, wherein the clamp comprises one of a key and tightening ball configured to effect the holding of the device on the patient's body.

5. The device of claim 2, wherein the connection element comprises markings for determining length between the fixation elements connected to the connection element.

6. The device of claim 2, wherein the fixation elements are integrated with stud elements and the connection element is of fixed length and has one or more slots or apertures therein along a length thereof with the slots or apertures being configured for insertion of the stud elements therein for variable position connection of the fixation elements to the connection element.

7. The device of claim 2, wherein the fixation elements are integrated with wheel members and the connection element comprises a track member with the wheel members being configured to be movable within the track member to provide the desired distance between the fixation elements.

8. The device of claim 2, wherein the fixation elements are fixedly attached to the connection element and the connection element is configured to be varied in length between fixation elements and locked into a desired length position.

9. The device of claim 8, wherein the connection element is comprised of at least two members pivotably attached to each other whereby length of the connection element is configured to be changed with such pivoting.

10. The device of claim 9, wherein the pivotal attachment is a hinge.

11. The device of claim 8, wherein the connection element is comprised of multiple connected elements, the total length of connected elements providing the desired distance between fixation elements.

12. The device of claim 8, wherein the connection element comprises slidably nested members which are configured to telescope to provide the desired distance between fixation elements.

13. The device of claim 8, wherein the connection element comprises a variable positioned flexible tube.

14. The device of claim 2, wherein the connection element is provided with at least one of a neck or body sling configured to facilitate support of the device on the patient.

15. A method of one of splinting, fixation or restraining of body parts or limbs or providing traction support thereof, comprising using the device of claim 1 with the steps of:
connecting at least two fixation elements to a connection element,
adjusting the length of the connection element between the fixation element to a desired length sufficient to maintain body parts or limbs apart from each other or restrained adjacent each other against movement away from the desired length,
engaging the fixation elements with body parts or limbs to be splinted, fixed or restrained against movement, and locking the fixation elements and connection elements against undesired relative movement.
